(19) United States
(12) Patent Application Publication
(10) Pub. No.: US 2026/0256892 A1
(43) Pub. Date: Sep. 3, 2026

(54) COLLAGEN-CONTAINING COMPOSITION DERIVED FROM FISH CARTILAGE

(71) Applicant: LINISE CO., LTD., Sapporo-shi, Hokkaido (JP)

(72) Inventors: Hideharu NAKANO, Hokkaido (JP); Masaki NARUMI, Hokkaido (JP); Hiroyuki SASAKI, Hokkaido (JP); Shoshi MIZUTA, Fukui (JP)

(73) Assignee: LINISE CO., LTD., Sapporo-shi, Hokkaido (JP)

(21) Appl. No.: 18/993,736

(22) PCT Filed: Jul. 14, 2023
(86) PCT No.: PCT/JP2023/026006

§ 371 Date: Jan. 13, 2025

(30) Foreign Application Priority Data
Jul. 14, 2022 (JP) 2022-113413

(51) Int. Cl. A61K 38/39; A61P 19/02

(52) U.S. Cl. CPC A61K 38/39; A61P 19/02

(57) ABSTRACT

There is provided a technique for effectively using a collagen-containing composition derived from fish cartilage. A cartilage-protecting composition or knee-joint-protecting composition that contains a collagen-containing composition derived from fish cartilage as an active ingredient, the collagen-containing composition containing type II collagen and type XI collagen. The collagen-containing composition preferably has a function for promoting hyaluronic acid production capabilities in cartilage cells. Additionally, the collagen-containing composition is preferably derived from salmon nose cartilage.

### TECHNICAL FIELD

The present invention relates to a technique for effectively using a collagen-containing composition derived from fish cartilage.

### BACKGROUND ART

Collagen is a protein that is generally widely distributed among animals such as mammals, birds, and fish, and that functions as a main constituent ingredient of extracellular matrices in these animals. In humans, collagen accounts for about 25-30% of the total mass of proteins. Three peptide chains having a molecular weight of about 100,000 are gathered together and a triple-helix structure is formed by hydrogen bonds as the molecular structure of the collagen. Triple-helix structural units therein are cross-linked in a telopeptide region accounting for end sections of the collagen molecules, and a higher-order structure such as a fibrous structure or a mesh structure is formed. There have been confirmed to be as many as 28 existing types of collagen in humans, these types being classified by function. For example, in type I collagen, which is the main protein in bones, the dermis, tendons, and the like, two α1 chains and one α2 chain form the triple-helix structure, the resulting fibrous structure having exceptional tensile strength, and this type of collagen functioning as a structural protein that maintains the form of the body and internal organs. In type II collagen, three α1 chains form the triple-helix structure, this type of collagen being localized in cartilage and the like and having a function as a structural protein similar to type I collagen. Type III and type V collagen coexist with type I collagen in skin and the like, and type XI collagen coexists with type II collagen in cartilage and the like. It is thought that type III, type V, and type XI collagen supplementarily participate in formation of collagen fibers that are suitable for the respective tissues (refer to Non-patent Documents 1 and 2). It is also thought that three types of chains, specifically an α1 chain, an α2 chain, and an α3 chain, form a triple-helix structure as the main molecular structure of type XI collagen (Non-patent Document 3).

As pertains to usage of collagen ingredients, type I collagen, which accounts for the majority of collagen in animal tissues, is widely used as a raw material in cosmetics, health foods, medical and pharmaceutical products, and the like. In recent years, use of type II collagen, which is localized in cartilage, has been increasing. For example, in Non-patent Document 4, it is reported that an antibody titer for type II collagen was elevated in the blood of patients with rheumatoid arthritis, and that immune tolerance resulting from orally administering non-denatured type II collagen makes it possible to anticipate an effect for preventing or treating rheumatoid arthritis (Non-patent Document 4).

Generally, methods for solubilizing and extracting collagen from animal tissues include methods involving, inter alia, a heating denaturation treatment, a solubilization treatment carried out using an acid or an alkali, or solubilization carried out using an enzyme treatment. However, in heating denaturation treatments, the triple chains may be loosened and randomly denatured. Additionally, in acid solubilization and alkali solubilization, productivity is poor because only a very small amount of collagen is eluted under ordinary non-denaturation conditions. In this, it is thought that using pepsin, which is a digestive enzyme, or the like makes it possible to partially decompose only a portion of the cross-linked structure and efficiently extract the triple-helix structural units.

In relation to methods for formulating a type II collagen ingredient, e.g., Patent Document 1 indicates that cow scapular cartilage was used as a raw ingredient, impurities in the raw material were removed by a prescribed pre-treatment in which a high-pressure water current was used, and then type II collagen having antigen-antibody reactivity with respect to serum from rheumatism patients was formulated by demineralization and pepsin solubilization treatments.

### RELATED-ART DOCUMENTS

### Non-Patent Documents

[Non-patent Document 1] Hattori, Shunji. “Collagen as an Animal Derived Fibrous Protein-its Character and Application.” Journal of The Society of Fiber Science and Technology, Japan, vol. 65, no. 12 (2009), pp. 453-461.
[Non-patent Document 2] Hattori, Shunji. “Collagen. Molecular Assemble and its Application.” The Society of Polymer Science, Japan, vol. 47, no. 6 (1998), pp. 394-397.
[Non-patent Document 3] Yoshioka, Hidekatsu. “Type XI collagen α1-chain gene: Primary structure of α1 chain, gene expression thereof, and regulation mechanism therefor”. Connective Tissue, vol. 29 (1997), pp. 39-47.
[Non-patent Document 4] Trentham, David E. et al. “Effects of oral administration of type II collagen on rheumatoid arthritis.” Science 261 (1993), pp. 1727-1730.

### Patent Documents

[Patent Document 1] Japanese Laid-Open Patent Application No. 2001-112419

### DISCLOSURE OF THE INVENTION

### Problems the Invention is Intended to Solve

However, although various formulation methods for extracting type II collagen, which is abundantly present among collagens derived from cartilage, in a non-denaturing manner have been developed in the previous art, only small amounts of type XI collagen, which is only sparsely present among cartilage collagens, are formulated and used in experimentation and research, and at present type XI collagen is not effectively used to the same extent as type II collagen.

It is an object of the present invention to provide a technique for enabling type XI collagen to be industrially produced as an ingredient that can be effectively applied in industrial settings and for using the type XI collagen.

### [Means for Solving the Aforementioned Problems]

As a result of thorough investigations for the purpose of achieving the aforementioned object, the inventors perfected the present invention upon discovering that using fish cartilage as an extraction raw material, solubilizing collagen using an acidic protease treatment, eluting the collagen into a liquid portion, and then adequately salting out the liquid portion makes it possible to more efficiently recover type XI collagen in addition to type II collagen.

Specifically, the present invention provides a cartilage-protecting composition that contains a collagen-containing composition derived from fish cartilage as an active ingredient, the collagen-containing composition containing type II collagen and type XI collagen.

The present invention also provides a knee-joint-protecting composition that contains a collagen-containing composition derived from fish cartilage as an active ingredient, the collagen-containing composition containing type II collagen and type XI collagen.

In the aforementioned compositions, the collagen-containing composition is preferably provided with functionality for promoting hyaluronic acid production capabilities in cartilage cells.

In the aforementioned compositions, the collagen-containing composition is preferably formulated so that the mass ratio of the type II collagen content and the type XI collagen content is 10:1 to 1:10, and that the collagen content per solid fraction is 30 mass % or greater.

In the aforementioned compositions, the collagen-containing composition is preferably derived from salmon nose cartilage.

### Effect of the Invention

According to the present invention, it is possible to provide a high-function collagen ingredient in which fish cartilage is used as an extraction raw material and which is useful as an ingredient to be blended into a food product, a supplement, a medical or pharmaceutical product, a medical ingredient, or the like.

### BRIEF DESCRIPTION OF THE DRAWINGS

FIG. 1 is a step chart showing one embodiment of a method for formulating a collagen-containing composition used in the present invention;

FIG. 2 is a step chart showing another embodiment of a method for formulating the collagen-containing composition used in the present invention;

FIG. 3 is a step chart showing yet another embodiment of a method for formulating the collagen-containing composition used in the present invention;

FIG. 4 is a flow chart of production carried out in production example 1;

FIG. 5 contains diagrams showing results of investigating, in test example 1, the solubilization rate from when collagen is eluted from a salmon nose cartilage raw material using an enzyme treatment of rhizopuspepsin, where FIG. 5(a) is a table in which values for cartilage weight, collagen content, solubilized fraction amount, solubilization rate, average solubilization rate, and standard deviation thereof are collected with respect to test materials in specimen nos. 1 to 3, FIG. 5(b) is a graph representing a change over time in the solubilization rate, and FIG. 5(c) is a table in which numeric values leading to the graph are collected;

FIG. 6 is a diagram showing a photograph of a gel from when a portion of a salting-out precipitate obtained in production example 1 is subjected to SDS-PAGE gel electrophoresis in test example 2;

FIG. 7 contains diagrams showing results from when a portion of the salting-out precipitate obtained in production example 1 is subjected to ion chromatography analysis in test example 3, where FIG. 7(a) is a diagram showing a photograph of a gel from when SDS-PAGE gel electrophoresis is carried out for each fractionated fraction produced using ion chromatography, FIG. 7(b) is a diagram showing an enlarged photograph of the portions of the gel including fraction no. 37 and fraction no. 41 in the SDS-PAGE gel electrophoresis, and FIG. 7(c) is a diagram showing an analysis chart for an ion chromatograph;

FIG. 8 contains diagrams showing results of analyzing, in test example 4, the salting-out precipitate, an 11% ammonium sulfate precipitate, and a 20% ammonium sulfate precipitate obtained in production example 1, where FIG. 8(a) is a diagram showing a photograph of a gel from when portions of the 20% ammonium sulfate precipitate and the salting-out precipitate are subjected to SDS-PAGE gel electrophoresis, and FIG. 8(b) is a table showing results obtained by measuring the hydroxyproline content of the salting-out precipitate, the 11% ammonium sulfate precipitate, and the 20% ammonium sulfate precipitate using dimethylbenzaldehyde colorimetry and multiplying the measured hydroxyproline content by a conversion coefficient for conversion to collagen content to derive the collagen content;

FIG. 9 is a graph showing results of investigating, in test example 5, the amount of hyaluronic acid produced from rabbit cartilage cells;

FIG. 10 contains graphs showing results of hierarchical analysis, in test example 6, of VAS scores for test subjects aged 50 or older, excluding test subjects for whom the score is 10 or lower; and

FIG. 11 contains graphs showing results of hierarchical analysis, in test example 6, of surveys for actions that are repeated ten times by test subjects aged 50 or older, excluding test subjects who did not experience any sense of discomfort during the actions.

### MODE FOR CARRYING OUT THE INVENTION

A collagen-containing composition used in the present invention can be formulated using fish cartilage as a basic source of collagen. There is no particular limitation as to the type of fish, the site of the cartilage tissue therein, and the like. Examples include salmon nose cartilage (salmon head cartilage), shark cartilage, ray cartilage, and squid cartilage. Salmon nose cartilage (salmon head cartilage) is particularly preferred due to having a high collagen content and being inexpensively procurable as a site that is ordinarily discarded in the field of processed seafood products. For example, because the heads from landed salmon are discarded in bulk in salmon roe processing and salmon fillet processing, it is possible to procure the heads and retrieve nose cartilage from the heads.

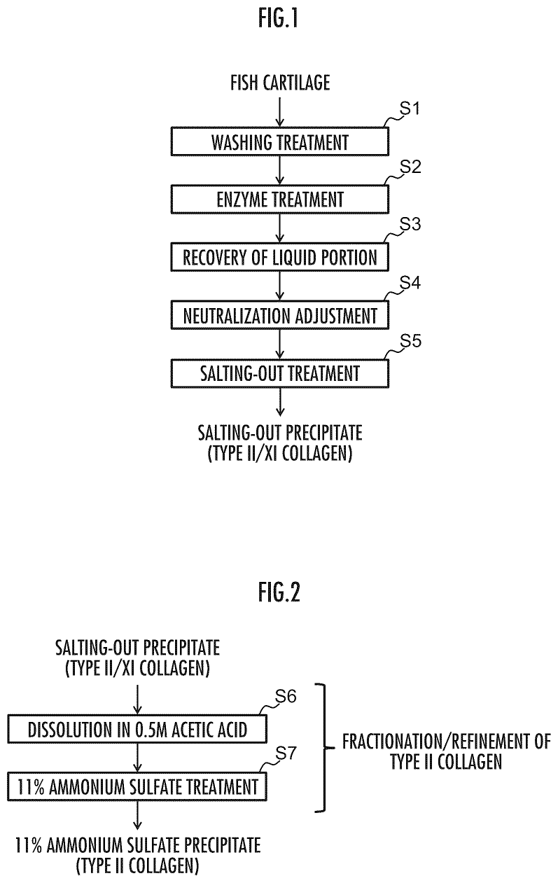

FIG. 1 shows one embodiment of a method for formulating the collagen-containing composition used in the present invention. In this embodiment, a collagen-containing composition that contains type II collagen and type XI collagen, which are collagens derived from cartilage, is obtained using fish cartilage as an extraction raw material. Specifically, the method for formulating the collagen-containing composition includes implementing: a step (indicated by “S1” in FIG. 1; referred to below as an “alkali washing step”) for performing a treatment for washing the raw material using an alkaline solvent; a step (indicated by “S2” in FIG. 1; referred to below as an “enzyme treatment step”) for performing, on a washed treated article after the alkali washing, an enzyme treatment carried out using an acidic protease in a weakly acidic solvent; a step (indicated by “S3” in FIG. 1; referred to below as a “collagen-containing liquid portion recovery step”) for subjecting an enzyme-treated article after the enzyme treatment to solid-liquid separation and recovering a collagen-containing liquid portion; a step (indicated by “S4” in FIG. 1; referred to below as a “collagen-containing liquid portion neutralization step”) for adjusting the pH of the collagen-containing liquid portion recovered through solid-liquid separation toward neutrality; and a step (indicated by “S5” in FIG. 1; referred to below as a “salting-out treatment step”) for salting out a neutralized treated article after the pH of the collagen-containing liquid portion is adjusted toward neutrality.

In the aforementioned formulation method, it is preferable to use, e.g., an article obtained by pulverizing fish cartilage and evening out the grain diameter thereof as the extraction raw material, although the present invention is not particularly limited thereto. The grain diameter of the cartilage raw material can be made uniform by, inter alia, performing a fragmentation treatment using a meat grinder for which there is used a plate having a prescribed hole diameter (e.g., a hole diameter of about 1.8-10 mm), or a homogenizer, a mass corroder, or the like. Evening out the grain diameter makes it possible to more efficiently perform alkali washing and elute the collagen. The grain diameter of the collagen raw material may typically be, e.g., within the range of 0.1-10 mm, within the range of 0.5-5 mm, or within the range of 1-3 mm.

In the alkali washing step of the aforementioned formulation method, a washing treatment is performed using an alkaline solvent on the fish cartilage that is the extraction raw material, and carbohydrate-based extracellular matrix substances such as proteoglycan and hyaluronic acid are removed from the extraction raw material. The alkaline solvent to be used is not particularly limited and may be a NaOH aqueous solution having a concentration within the range of 0.01-1M, a NaOH aqueous solution having a concentration within the range of 0.05-0.5M, or a NaOH aqueous solution having a concentration within the range of 0.1-0.4M.

The alkali washing step is preferably performed by stirring the fish cartilage raw material for a prescribed time in the alkaline solvent and then performing solid-liquid separation. This makes it possible to more effectively remove impurities such as carbohydrate-based extracellular matrix substances from the fish cartilage raw material. There is no particular limitation as to temperature conditions in stirring for the purpose of such alkali washing. However, the temperature is preferably managed so as to be low. For example, the temperature may be within the range of 0-12° C., within the range of 2-10° C., or within the range of 4-8° C. There is also no particular limitation as to the stirring time in stirring for the purpose of the alkali washing. For example, the stirring time may be within the range of 30 minutes to 48 hours, within the range of 1-36 hours, or within the range of 3-24 hours. The solid-liquid separation may be performed using, e.g., a draining basket or filter cloth having a mesh size of about 60-100, or centrifugal separation.

According to a given aspect, the alkali washing treatment for the fish cartilage raw material that is performed using an alkaline solvent in the alkali washing step may be performed a plurality of times (e.g., two to four times) by performing solid-liquid separation of the alkaline solvent after the stirring for a prescribed time, removing the alkaline solvent, subsequently adding a new alkali solvent, and repeating the washing. This makes it possible to even more effectively remove impurities such as carbohydrate-based extracellular matrix substances from the fish cartilage raw material. Additionally, after the alkali washing, a water washing treatment may be performed by adding water and performing solid-liquid separation after stirring for a prescribed time. The water washing treatment may also be repeated a plurality of times (e.g., two to four times). It is preferable to repeat the water washing treatment until the pH of the washing liquid used therein is near neutral. This makes it possible to prevent the alkali that is used from affecting subsequent treatments.

In the enzyme treatment step of the aforementioned formulation method, an enzyme treatment carried out using an acidic protease in a weakly acidic solvent is performed on the washed treated article after the alkali washing step, and collagen is solubilized. The acidic protease to be used is not particularly limited. Examples thereof include pepsin, and non-animal pepsin derived from fungi is particularly preferred because it is not necessary to use an ingredient derived from an animal that imparts an impression of illness such as bovine spongiform encephalopathy or foot-and-mouth disease. Examples of non-animal pepsin derived from fungi include rhizopuspepsin, which is an acidic protease produced by Rhizopus niveus, a type of mold. The weakly acidic solvent to be used also is not particularly limited and may be, e.g., an acetic acid aqueous solution having a concentration within the range of 10-500 mM an acetic acid aqueous solution having a concentration within the range of 250-500 mM, or an acetic acid aqueous solution having a concentration within the range of 400-500 mM. Alternatively, the weakly acidic solvent may be, e.g., a citric acid aqueous solution having a concentration within the range of 10-500 mM, a citric acid aqueous solution having a concentration within the range of 50-500 mM, or a citric acid aqueous solution having a concentration within the range of 400-500 mM. In this, the pH must be adjusted toward acidity for the purpose of achieving ideal conditions for the acidic protease.

In the enzyme treatment, the ideal pH conditions differ depending on the type of enzyme used, and there are no generalized rules therefor. However, the pH may be within the range of 1-6, within the range of 2-5, or within the range of 2.5-3.5. In the enzyme treatment, the ideal temperature conditions differ depending on the type of enzyme used, and there are no generalized rules therefor. However, the temperature is preferably within a range in which collagen is not denatured, the temperature optionally being, e.g., within the range of 0-12° C., within the range of 2-10° C., or within the range of 4-8° C. There is no particular limitation as to the treatment time in the enzyme treatment. However, the treatment time may be, e.g., within the range of 30 minutes to 48 hours, within the range of 1-36 hours, or within the range of 3-24 hours. When the aforementioned rhizopuspepsin is used as the acidic protease, it is typical to add the rhizopuspepsin so that the amount of the enzyme added is 1/100-⅕ with respect to the solid fraction (dry weight) of the introduced fish cartilage raw material, or 1/50- 1/10 depending on the situation, or even 1/25- 1/15 depending on the situation, and it is typical to set the concentration of a rhizopuspepsin preparation incorporated into the weakly acidic solvent to 0.001-1 mass %, or 0.005-0.1 mass % depending on the situation, or even 0.01-0.05 mass % depending on the situation.

According to a given aspect, the pH in the enzyme treatment step may be adjusted toward acidity in advance by adding the weakly acidic solvent to the fish cartilage raw material and carrying out treatment for a prescribed time before the acidic protease is added. This makes it possible to prevent the pH from shifting toward alkalinity and prevent the efficiency of collagen solubilization from deteriorating during the treatment carried out using the acidic protease. It is possible to adjust the pH toward acidity by adding the weakly acidic solvent in an amount that is, e.g., 1-50 times the amount of the cartilage raw material, or 3-20 times the amount of the cartilage raw material depending on the situation, or even 5-10 times the amount of the cartilage raw material depending on the situation, and then carrying out a stirring treatment for a prescribed time, although the present invention is not limited to this process. There is no particular limitation as to the temperature conditions in adjusting the pH toward acidity. However, the temperature is preferably managed so as to be low. For example, the temperature may be within the range of 0-12° C., within the range of 2-10° C., or within the range of 4-8° C. There is also no particular limitation as to the stirring time in adjusting the pH toward acidity. For example, the stirring time may be within the range of 30 minutes to 48 hours, within the range of 1-36 hours, or within the range of 3-24 hours.

In the collagen-containing liquid portion recovery step of the aforementioned formulation method, the enzyme-treated article after the enzyme treatment step is subjected to solid-liquid separation, and a collagen-containing liquid portion eluted from the fish cartilage raw material is recovered. The solid-liquid separation may be performed using, e.g., a centrifugal separator. In the collagen-containing liquid portion neutralization step, the pH of the recovered liquid portion is adjusted toward neutrality so as to be near neutral. This adjustment toward neutrality facilitates effective recovery of not only type II collagen, which is abundantly present as cartilage-derived collagen, but also type XI collagen, which is a sparse type of cartilage-derived collagen, through the subsequent salting-out treatment. The adjustment toward neutrality can be performed by, inter alia, using, e.g., a 0.1-1M HCl aqueous solution or a 0.1-1M NaOH aqueous solution, and adding this acid or base to the recovered collagen-containing liquid portion, as appropriate, while measuring the pH, although the present invention is not limited thereto. The pH is preferably adjusted to within the range of, e.g., 7.5-8.5, and is particularly preferably near 8.

In the salting-out treatment step of the aforementioned formulation method, the neutralization treated article after the collagen-containing liquid portion neutralization step is salted out, and type II collagen and type XI collagen are recovered as a salting-out precipitate. The salting out may be performed using an ordinary method, and specifically can be performed by adding sodium chloride or another salt that is solid or in another form suited to salting out, subsequently stirring for a prescribed time, and then performing solid-liquid separation. The final concentration of the salt may be, e.g., within the range of 2-5M, within the range of 2.5-4.5M, or within the range of 3-4M. The final concentration of the salt is particularly preferably 4M or greater, and particularly is near 4.4M. There is no particular limitation as to the temperature conditions in salting out. However, the temperature is preferably managed so as to be low. For example, the temperature may be within the range of 0-12° C., within the range of 2-10° C., or within the range of 4-8° C. There is also no particular limitation as to the stirring time in salting out. For example, the stirring time may be within the range of 30 minutes to 48 hours, within the range of 1-36 hours, or within the range of 3-24 hours. The salting-out precipitate may be recovered using the same solid-liquid separation as is described above. For example, a centrifugal separator may be used.

Salts can be removed from the salting-out precipitate obtained as described above through ultrafiltration, dialysis, or the like. The salting-out precipitate may be subjected to vacuum freeze-drying to formulate a dried article. Examples of methods for removing salts from the salting-out precipitate include: methods performed using ultrafiltration in which a hollow fiber module, a ceramic filter, a flat-membrane-type filter, or the like is used; and methods for filling the salting-out precipitate into a dialysis tube and immersing the dialysis tube in water to carry out desalination.

The collagen content of the collagen-containing composition, which contains the type II collagen and the type XI collagen obtained as described above, may be 30 mass % or greater, 40 mass % or greater, 50 mass % or greater, 60 mass or greater, 65 mass % or greater, 70 mass % or greater, 75 mass % or greater, 80 mass % or greater, 85 mass % or greater, 90 mass or greater, or 95 mass % or greater per solid fraction. The mass ratio of the type II collagen content and the type XI collagen content may be within the range of 10:1 to 1:10, within the range of 8:2 to 2:8, or within the range of 7:3 to 3:7. The collagen content can be derived by performing an acid hydrolysis treatment of the collagen, subsequently analyzing the amount of hydroxyproline using amino acid composition analysis or dimethylbenzaldehyde colorimetry, and multiplying the measured amount of hydroxyproline by a prescribed conversion coefficient for conversion to an amount of collagen. For example, in the case of salmon collagen, hydroxyproline (Hyp) accounts for 4.3 residues among the 1000 residues of amino acids, the Hyp content is 7.92%, and the conversion coefficient for conversion from the amount of Hyp to the amount of collagen is 12.63.

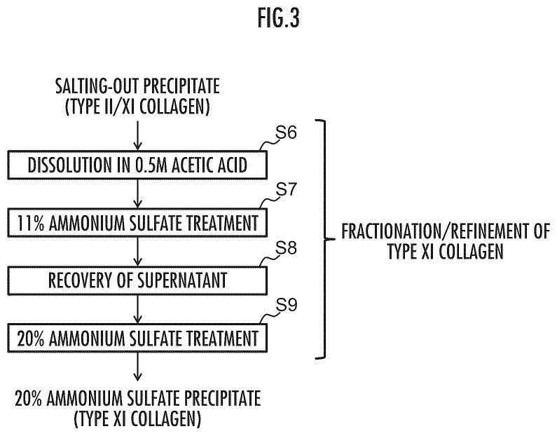

FIG. 2 shows another embodiment of a method for formulating the collagen-containing composition used in the present invention. In this embodiment, the method includes implementing a step (indicated by “S6” in FIG. 2) for dissolving the above-described salting-out precipitate that contains type II collagen and type XI collagen in 0.5M acetic acid, and a step (indicated by “S7” in FIG. 2) for performing an ammonium sulfate treatment using ammonium sulfate at a prescribed final concentration. A type-II-collagen-containing composition in which the type II collagen is made more abundant is thereby obtained as an ammonium sulfate precipitate. In the drawings, 0.5M acetic acid is given as an example of the acid solvent in the dissolution treatment (S6), but the concentration may be appropriately set so that the pH of the solution is within the range of 2.6-2.8 by using, e.g., 0.4-0.6M acetic acid or 0.4-0.6M citric acid. The ammonium sulfate treatment can be performed using an ordinary method, and specifically can be performed by adding ammonium sulfate that is solid or in another form suited to salting out, subsequently stirring for a prescribed time, and then performing solid-liquid separation. In the drawings, a final concentration of 11 w/v % is given as an example of the final concentration of the ammonium sulfate in the ammonium sulfate treatment (S7), but this final concentration may be, e.g., within the range of 10-13 w/v % or within the range of 10.5-12.5 w/v %. The final concentration of the ammonium sulfate is particularly preferably near 11 w/v %. There is no particular limitation as to the temperature conditions in the ammonium sulfate treatment. For example, the temperature may be within the range of 0-12° C., within the range of 2-10° C., or within the range of 4-8° C. There is also no particular limitation as to the stirring time in the ammonium sulfate treatment. For example, the stirring time may be within the range of 30 minutes to 48 hours, within the range of 1-36 hours, or within the range of 3-24 hours. The ammonium sulfate precipitate may be recovered using the same solid-liquid separation as is described above. For example, a centrifugal separator may be used.

Salts can be removed from the ammonium sulfate precipitate obtained as described above through using ultrafiltration, dialysis, or the like. The ammonium sulfate precipitate may be subjected to vacuum freeze-drying to formulate a dried article. Examples of methods for removing salts from the ammonium sulfate precipitate include: physical desalination carried out using ultrafiltration in which a hollow fiber module, a ceramic filter, a flat-membrane-type filter, or the like is used; and methods for filling the ammonium sulfate precipitate into a dialysis tube and immersing the dialysis tube in water to carry out desalination.

The collagen content of the type-II-collagen-containing composition in which the type II collagen is made more abundant, which is obtained as described above, may be 30 mass % or greater, 40 mass % or greater, 50 mass % or greater, 60 mass % or greater, 65 mass % or greater, 70 mass % or greater, 75 mass % or greater, 80 mass % or greater, 85 mass % or greater, 90 mass % or greater, or 95 mass % or greater per solid fraction.

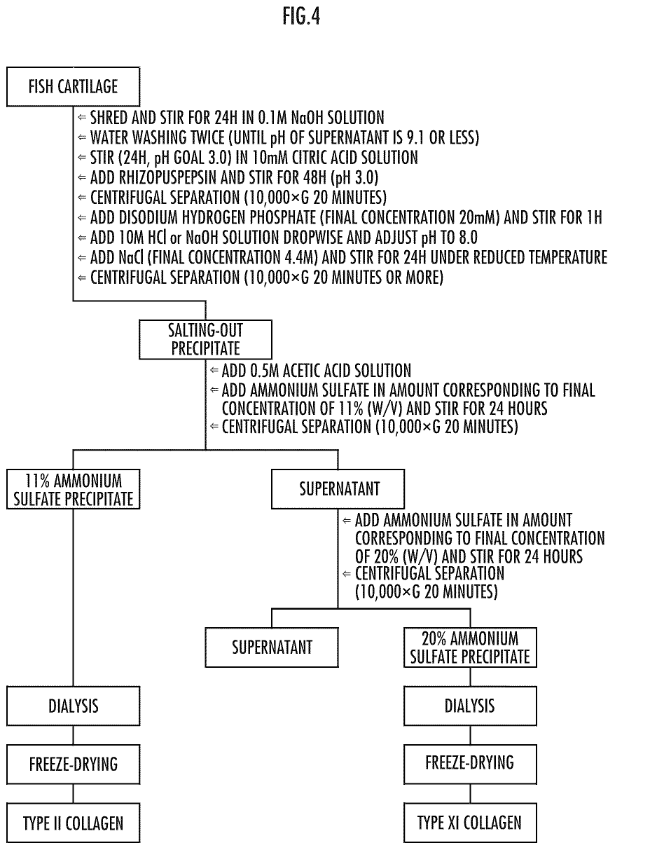

FIG. 3 shows yet another embodiment of a method for formulating the collagen-containing composition used in the present invention. In this embodiment, the method includes implementing a step (indicated by “S6” in FIG. 3) for dissolving the above-described salting-out precipitate that contains type II collagen and type XI collagen in 0.5M acetic acid, a step (indicated by “S7” in FIG. 3) for performing an ammonium sulfate treatment using ammonium sulfate at a prescribed first final concentration, a step (indicated by “S8” in FIG. 3) for recovering supernatant after the ammonium sulfate treatment, and a step (indicated by “S9” in FIG. 3) for performing an ammonium sulfate treatment using ammonium sulfate at a prescribed second final concentration. A type-XI-collagen-containing composition in which the type XI collagen is made more abundant is thereby obtained as an ammonium sulfate precipitate. As indicated above, in the drawings, 0.5M acetic acid is given as an example of the acid solvent in the dissolution treatment (S6), but the concentration may be appropriately set so that the pH of the solution is within the range of 2.6-2.8 by using, e.g., 0.4-0.6M acetic acid or 0.4-0.6M citric acid. The ammonium sulfate treatment can be performed using an ordinary method, and specifically can be performed by adding ammonium sulfate that is solid or in another form suited to salting out, subsequently stirring for a prescribed time, and then performing solid-liquid separation. The first final concentration of the ammonium sulfate in the ammonium sulfate treatment (S7) may be, e.g., within the range of 10-13 w/v % or within the range of 10.5-12.5 w/v %. The first final concentration of the ammonium sulfate is particularly preferably near 11 w/v %. Additionally, the second final concentration of the ammonium sulfate in the ammonium sulfate treatment may be, e.g., within the range of 18-22 w/v %, or within the range of 19-21 w/v %. The second final concentration of the ammonium sulfate is particularly preferably near 20 w/v %. There is no particular limitation as to the temperature conditions in the ammonium sulfate treatment. For example, the temperature may be within the range of 0-20° C., within the range of 2-15° C., or within the range of 4-10° C. There is also no particular limitation as to the stirring time in the ammonium sulfate treatment. For example, the stirring time may be within the range of 30 minutes to 48 hours, within the range of 1-36 hours, or within the range of 3-24 hours. The supernatant or precipitate after the ammonium sulfate treatment may be recovered using the same solid-liquid separation as is described above. For example, a centrifugal separator may be used.

Salts can be removed from the ammonium sulfate precipitate obtained as described above through using ultrafiltration, dialysis, or the like. The ammonium sulfate precipitate may be subjected to vacuum freeze-drying to formulate a dried article. Examples of methods for removing salts from the ammonium sulfate precipitate include: physical desalination carried out using ultrafiltration in which a hollow fiber module, a ceramic filter, a flat-membrane-type filter, or the like is used; and methods for filling the ammonium sulfate precipitate into a dialysis tube and immersing the dialysis tube in water to carry out desalination.

The collagen content of the type-XI-collagen-containing composition in which the type XI collagen is made more abundant, which is obtained as described above, may be 30 mass % or greater, 40 mass % or greater, 50 mass % or greater, 60 mass % or greater, 65 mass % or greater, 70 mass % or greater, 75 mass % or greater, 80 mass % or greater, 85 mass % or greater, 90 mass % or greater, or 95 mass % or greater per solid fraction.

The salting-out precipitate that contains type II collagen and type XI collagen, the 11% ammonium sulfate precipitate that contains type II collagen, and the 20% ammonium sulfate precipitate that contains type XI collagen, which are obtained through the formulation methods described above, may be dried using a decompression dryer, an atomization dryer, or another means that would be ordinarily well known to persons skilled in the art. The resulting dried article may furthermore be broken down, pulverized, or otherwise powderized together with being dried. If the precipitate is in the form of a dried article, then moisture will be removed therefrom in the same manner as with the dried article after the freeze-drying described above, thus preventing decomposition and the like and yielding exceptional storage stability. In drying, dextrin or another diluent, crystalline cellulose, silica, or the like may be added in a preparational manner.

For the purpose of powderization, a pulverizer, a mill, a mass corroder, or another means that would be ordinarily well known to persons skilled in the art can be used. As pertains to the granularity after powderization, it is preferable to carry out powderization to the extent that about 90 mass % or more of the entire substance passes through a mesh size of 30 (opening size: 500 μm), and it is more preferable to carry out powderization to the extent that about 90 mass % or more of the entire substance passes through a mesh size of 60 (opening size: 250 μm). Alternatively, it is preferable to carry out powderization to the extent that about 90 mass % or more of the entire substance passes through a screen having a hole diameter of 0.3 mm or greater and 0.75 mm or less.

The salting-out precipitate that contains type II collagen and type XI collagen, the 11% ammonium sulfate precipitate that contains type II collagen, and the 20% ammonium sulfate precipitate that contains type XI collagen, which are obtained through the formulation methods described above, may be mixed together in a desired blend as appropriate to form the collagen-containing composition used in the present invention.

As indicated in the examples (described later), a collagen-containing composition derived from fish cartilage that is provided by the present invention has an exceptional effect for promoting hyaluronic acid production capabilities in cartilage cells. The collagen-containing composition also has an exceptional effect for protecting knee joints. Thus, it is possible to form a functional composition that contains the collagen-containing composition as an active ingredient. For example, a composition for protecting cartilage can be provided as a functional composition. Additionally, for example, a composition for protecting knee joints can be provided as a functional composition. According to a given aspect, the functionality that is provided by the present invention may be functionality as an agent for promoting regeneration of joint cartilage cells, functionality as an agent for promoting propagation of cartilage cells, or functionality as an agent for promoting turnover of cartilage cells. The functional composition may be a composition for use by able-bodied persons, particularly able-bodied persons aged 50 or older. The functional composition may also be a composition targeting pet animals and other animals.

There are no particular limitations in regard to whether other components, in addition to the type II collagen and/or the type XI collagen, are contained in the functional composition that contains the collagen-containing composition derived from fish cartilage as an active ingredient and that is provided by the present invention, provided that the other components do not adversely affect the objective of the functional composition. Examples of the other components include vitamin C, imidazole peptides, collagen peptides, salmon ovary exodermis peptides, and β-hydroxy-β-methylbutyric acid (HMB).

The use form of the functional composition that contains the collagen-containing composition derived from fish cartilage as an active ingredient and that is provided by the present invention is not particularly limited. In this, the functional composition is preferably used upon being formed in such a manner as to be orally ingested. In the case of an oral composition, examples of the use form include those in which the type II collagen and/or the type XI collagen is used without further modification, and the oral composition may, as necessary, have a tableted form (pill, tablet, chewable pill, oral disintegrant), a liquid form (liquid), a syrup form (syrup), a powdered form (granules, fine granules), a capsule form (capsule), a soft-capsule form (soft capsule), a solid form, a semi-liquid form, a cream form, or a paste form.

The ingestion amount may be set as appropriate in accordance with the health state or illness state of the human or animal to which the composition is applied, the purpose of ingestion, or the like, and there is no particular limitation as to the ingestion amount. For example, the ingestion amount per day may be, e.g., 5 mg or higher and 100 mg or lower, 5 mg or higher and 40 mg or lower, 5 mg or higher and 20 mg or lower, 10 mg or higher and 20 mg or lower, or 10 mg or higher and 15 mg or lower, in terms of collagen amount. As an ingestion period, the composition is preferably used so as to be ingested over the course of twelve weeks or longer. The ingestion period may be, e.g., two weeks, four weeks, six weeks, eight weeks, ten weeks, or twelve weeks, and the composition may be used so as to be ingested continuously or intermittently over these periods.

Uses of the collagen-containing composition derived from fish cartilage that is provided by the present invention can be directed to, e.g., health foods, functional foods, and other food products and supplements, medical and pharmaceutical products, medical ingredients, and the like, and the collagen-containing composition can be used particularly suitably as a raw-material ingredient therefor. Additionally, as mentioned above, the collagen-containing composition can be used not only for humans but also for pet animals and other animals.

### EXAMPLES

The present invention is specifically described below by way of production examples and test examples, but the scope of the present invention is in no way limited by these production examples and test examples.

### Production Example 1

Salmon heads discharged from a seafood processing plant were procured, nose cartilage was retrieved from the heads, and 200 g of raw cartilage was harvested (an amount corresponding to eight salmon). The harvested raw cartilage was subjected to a fragmentation treatment using a meat grinder to which was attached a plate having a hole diameter of 2.8 mm and used as an extraction start-off raw material.

Collagen was extracted from the extraction raw material formulated as described above.

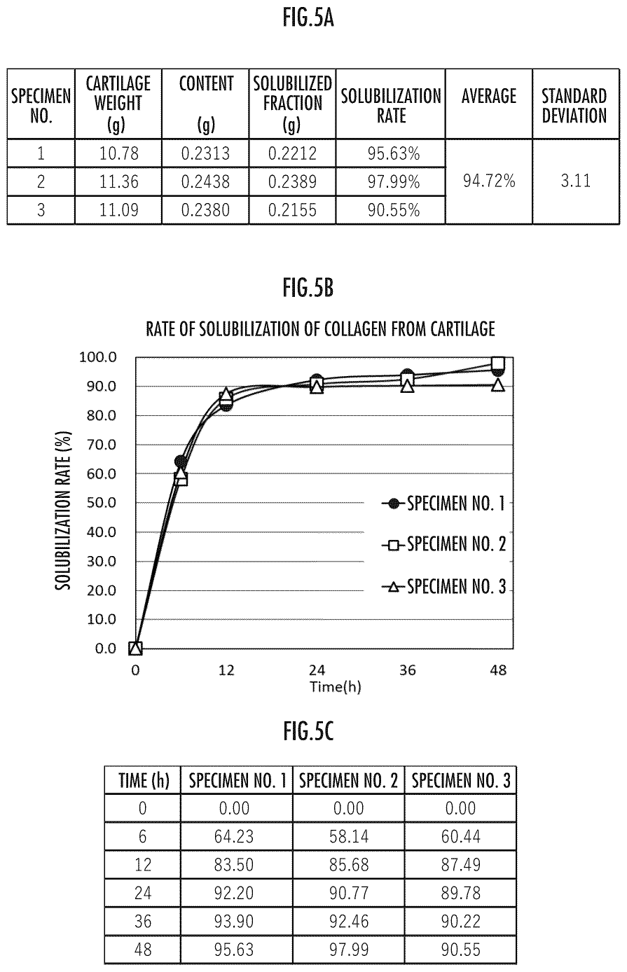

FIG. 4 shows a flow chart of production carried out in the present production example. Specifically, a composition was formulated as described below.
A 0.1M NaOH aqueous solution was added in an amount of 2400 mL to 200 g of the extraction raw material, the mixture was stirred for 24 hours at 4° C., the mixture was subsequently treated at 10,000 g×20 minutes using a centrifugal separation device to carry out solid-liquid separation, and the resulting solid portion was recovered.
Water was added in an amount of 2400 mL, the mixture was stirred at 4° C., the mixture was subsequently treated at 10,000 g×20 minutes using a centrifugal separation device to carry out solid-liquid separation, and the resulting solid portion was recovered, the same operations furthermore being repeated once more. Upon measurement of the pH of supernatant in the second instance, the pH was 11.92. The same process of water washing was furthermore repeated, and the pH at the time of the final water washing was 9.47.
A 10 mM citric acid aqueous solution was added in an amount of 2000 mL, the mixture was stirred for 24 hours at 4° C., and, upon measurement of the pH of supernatant, the pH was 3.0.
Rhizopuspepsin (trade name “Neurase F3G,” made by Amano Enzyme, Inc.) was added, and the mixture was stirred for 48 hours at 4° C. Upon measurement of the pH after this treatment, the pH was 3.0.
The mixture was treated at 10,000 g×20 minutes using a centrifugal separation device to carry out solid-liquid separation, and the resulting liquid portion was recovered.
Disodium hydrogen phosphate was added in such an amount as to reach a final concentration of 20 mM, and the mixture was stirred for 1 hour at 4° C.
The pH was adjusted to 8.0 through a fine adjustment carried out using dropwise addition of a 6M HCl aqueous solution or a 6M NaOH aqueous solution.
Powdered NaCl was added in such an amount as to reach a final concentration of 4.4M, and the mixture was stirred for 24 hours at 4° C.
The mixture was treated at 10,000 g×20 minutes using a centrifugal separation device to carry out solid-liquid separation, and the resulting solid portion was recovered as a salting-out precipitate.
A 0.5M acetic acid aqueous solution was added in an amount of 1200 mL, and the salting-out precipitate was dissolved.
Powdered ammonium sulfate was added in such an amount as to reach a final concentration of 11 W/V %, and the mixture was stirred for 24 hours at 4° C.
The mixture was treated at 10,000 g×20 minutes using a centrifugal separation device to carry out solid-liquid separation, and the resulting solid portion was recovered as an 11% ammonium sulfate precipitate. The liquid portion was recovered as supernatant.
Powdered ammonium sulfate was added to the supernatant in such an amount as to reach a final concentration of 20 W/V %, and the mixture was stirred for 24 hours at 4° C.
The mixture was treated at 10,000 g×20 minutes using a centrifugal separation device to carry out solid-liquid separation, and the resulting solid portion was recovered as a 20% ammonium sulfate precipitate.
Desalination carried out using dialysis was performed. Specifically, the resultant was filled into a dialysis tube (cellulose tube 36/32 made by Eidia Co., Ltd.) having a fractional molecular weight of 12,000-16,000 and the dialysis tube was immersed in water to carry out desalination.
The resulting material was dried using a vacuum freeze-dryer.

### Test Example 1

The rate at which collagen was solubilized using the enzyme treatment carried out using rhizopuspepsin in the production flow of production example 1 was examined.

First, the amount of collagen contained in the salmon nose cartilage used as the extraction raw material was measured. Specifically, 5 ml of 6M HCl (containing 0.04% mercaptoethanol) was added to 30 mg of freeze-dried cartilage to perform a decomposition treatment, the sample tube was sealed after the freeze-drying, a hydrolysis treatment was subsequently carried out at 110° C. for 24 hours, after which the sample was transferred to a round-bottomed flask, and the HCl was removed using an evaporator. Ultrapure water was added in an amount of 5 mL to the sample remaining at the bottom of the round-bottomed flask to obtain an analysis sample, and hydroxyproline (Hyp), which is unique to collagen, was quantified using an amino acid automatic analysis device.

The supernatant after the enzyme treatment in production example 1 was harvested over time, and the amount of collagen therein was measured. Specifically, 1 mL of the harvested enzyme-treated supernatant was dispensed into a sample tube, 1 mL of 12M HCl was added, the sample was sealed, and hydrolysis was carried out under heating for three hours using a hot block bath at 130° C. The sample was transferred to a round-bottomed flask, and hydrochloric acid was removed using an evaporator. Ultrapure water was added in an amount of 5 mL to the sample remaining at the bottom of the round-bottomed flask to obtain an analysis sample, after which Hyp, which is unique to collagen, was measured through colorimetry using dimethybenzaldehyde (J. F. Woesnner Jr., Archives of Biochemistry and Biophysics, 93, 440-447, 1961) in accordance with a normal method.

The Hyp content (g) was multiplied by a coefficient of 12.63 and thereby converted to the collagen content (g).

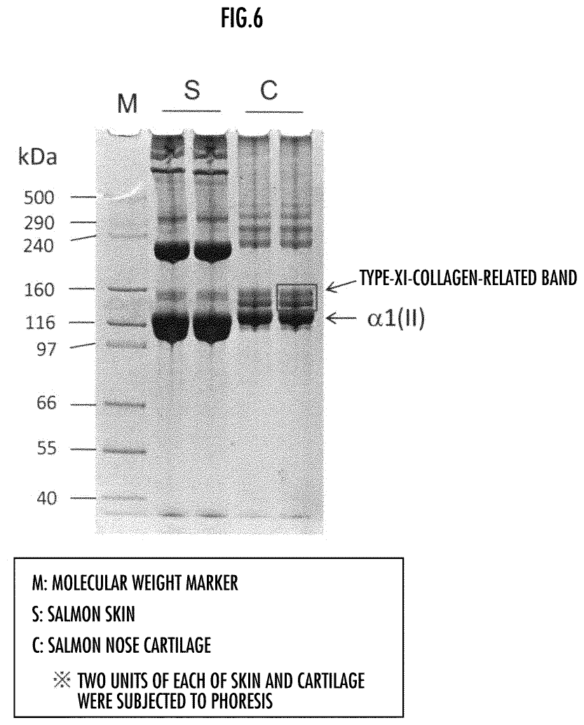

Once measurement had been carried out three times, all of the computed values stabilized at a high solubilization rate of 90% or higher, as shown in FIG. 5(a). Additionally, when the solubilization rate was observed over time, a solubilization rate of 90% or higher was achieved after 24 hours from the start of the enzyme treatment, as shown in FIGS. 5(b) and 5(c).

### Test Example 2

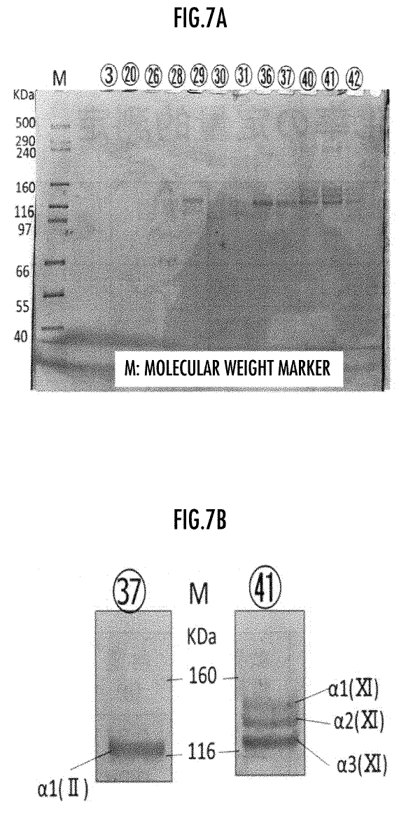

FIG. 6 shows a photograph of a gel from when a portion of the salting-out precipitate obtained in production example 1 is subjected to SDS-PAGE gel electrophoresis.

As shown in FIG. 6, a band near a molecular weight of 116 kDa that is characteristic of α1 (triple-helix chain) in type II collagen, which is a collagen derived from cartilage, was observed to a greater extent in a salting-out precipitate derived from salmon cartilage than in the electrophoretic pattern of the collagen sample derived from salmon skin. In addition, bands thought to be associated with α1 and α2 that are two types of chains characteristic of type XI collagen, which has a triple-helix structure of α1, α2, and α3, were observed near a molecular weight of 130-160 kDa.

### Test Example 3

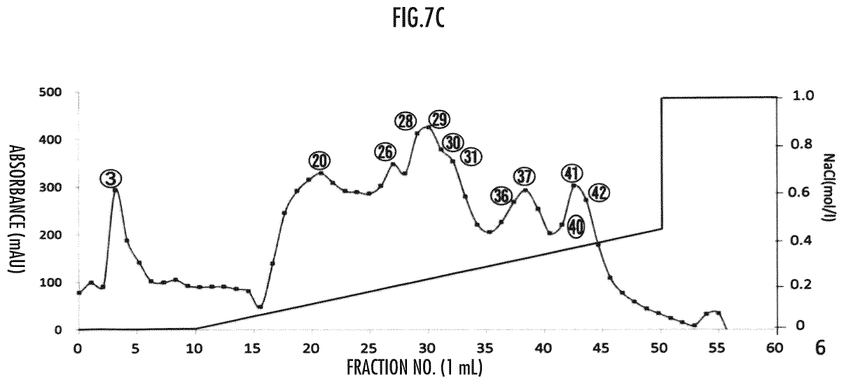

FIG. 7 shows results from when a portion of the salting-out precipitate obtained in production example 1 is subjected to ion chromatography analysis.

Specifically, a portion of the salting-out precipitate obtained in production example 1 was dissolved in a 20 mM sodium acetate buffer solution (pH 4.8) containing 2M urea, and the mixture was supplied to cation exchange chromatography. An ion exchange column (CM-825 made by Showa Denko KK) packed with a filler having carboxymethyl groups as ligands was used as a column. Adsorbed proteins were eluted by using an NaCl gradient (concentration gradient) up to 0-0.4M over 40 minutes at a flow rate of 1.0 mL/min. The light absorbance of liquid eluted from the column at a wavelength of 230 nm was continuously measured, whereby the eluted proteins were detected, and the eluted liquid was recovered using a fraction collector as 1 mL per fraction. The temperature of the column was maintained at 18° C. The resulting peak fractions were subjected to SDS-PAGE gel electrophoresis.

Components stained by Coomassie brilliant blue R-250, which is a protein stain that was applied to fraction nos. 31-42, emerged among emergent peaks produced by the absorbance of each fractionated fraction produced using ion chromatography in FIG. 7(c) with respect to a wavelength of 230 nm, and bands near a molecular weight of 116-160 kDa were observed for all of these components, as shown in FIG. 7(a). Additionally, a band thought to be produced by a triple chain of α1, which corresponds to type II collagen, emerged for fraction no. 37 produced using ion chromatography, and bands thought to be produced by a triple chain of α1 to α3, which corresponds to type XI collagen, emerged for fraction no. 41, as shown in FIG. 7(b).

### Test Example 4

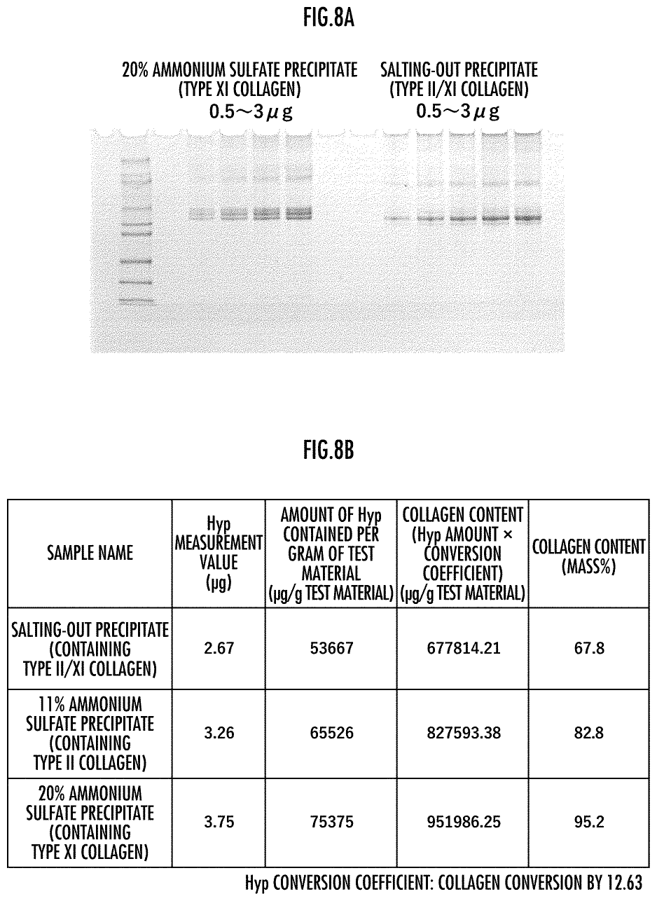

FIG. 8 shows results of analyzing the salting-out precipitate, the 11% ammonium sulfate precipitate, and the 20% ammonium sulfate precipitate obtained in production example 1.

As indicated by the results of SDS-PAGE gel electrophoresis in FIG. 8(a), the type XI collagen that emerged in fraction no. 41, which was fractionated in the ion chromatography analysis in test example 3, was included in the 20% ammonium sulfate precipitate obtained in production example 1, and the type II collagen that emerged in fraction no. 37 and the type XI collagen that emerged in fraction no. 41, which were fractionated in the ion chromatography analysis in test example 3, were included in the salting-out precipitate obtained in production example 1.

As indicated by the results of measuring the amount of collagen in FIG. 8(b), the collagen content of the salting-out precipitate obtained in production example 1 was 67.8 mass %, the collagen content of the 11% ammonium sulfate precipitate was 82.8 mass %, and the collagen content of the 20% ammonium sulfate precipitate was 95.2 mass %.

Separately, the salting-out precipitate obtained in production example 1 was subjected to SDS-PAGE gel electrophoresis and protein staining, and the resulting electrophoretic pattern was analyzed. Based on the assumption that, in descending order of molecular weight in the type XI collagen, attributions were made as α1 chains and α2 chains, the remaining α3 chains were included therewith in a ratio of 1:1:1 parts by mass, and the α3 chains would overlap the α1 chains in the type II collagen, with the staining strength of bands corresponding to the α1 chains and the α2 chains in type XI collagen having a purity of about 95% being used as an index, a conversion coefficient for converting the staining strength of the bands to collagen content was derived. Upon stoichiometrically applying the conversion coefficient to other bands as well and performing a rough quantitative analysis, it was estimated that type II collagen accounted for 60 mass % of the collagen contained in the salting-out precipitate obtained in production example 1, and that type XI collagen accounted for 40 mass % of said collagen.

### Test Example 5

The utility of the collagen-containing composition that contained the type II collagen and the type XI collagen was evaluated.

Specifically, hyaluronic acid production testing was implemented as described below using rabbit cartilage cells.

### [1. Substances Under Test]

Type I collagen (derived from cow epidermis) (MP Biomedicals, Inc.)
Type II collagen (derived from salmon nose cartilage) (Linise Co., Inc.)
Type II/type XI collagen [type II: 80 mass %; type XI: 20 mass %] (derived from salmon nose cartilage) (Linise Co., Inc.)

### [2. Cells/Medium]

Rabbit cartilage cells, joint cartilage from female Japanese white rabbits (“Cartilage cell culturing kit,” Cosmo Bio KK)
Cartilage cell culturing medium (“Cartilage cell culturing kit,” Cosmo Bio KK)
Cartilage cell differentiation medium (“Cartilage cell culturing kit,” Cosmo Bio KK)

### [3. Culturing]

Cells procured in a culturable state were cultured in a CO₂ incubator (5% CO₂, 37° C.) using a culturing medium belonging to the cartilage cell culturing kit, and the cultured cells were detached and cryopreserved the next day.

Separately, the type I collagen, the type II collagen, and the type II/type XI collagen serving as substances under test were each formulated using the cartilage cell differentiation medium so as to reach 10 mg/mL, and a filter was sterilized. The substances under test were formulated immediately before being added to the medium.

Thawed cells were revived in the culturing medium and cultured in a T-75 flask. The cells were detached, after which a sample having a cell concentration of 2×10⁵ cells/mL was formulated using the differentiation medium to which the substances under test were added. The sample was sown in 1.5-mL microtubes and cultured at 5% CO₂ and 37° C. using the CO₂ incubator. The medium was replaced every two to three days. Culturing was performed for two and a half weeks (17 days), and the final instance of medium replacement (500 μL/tube) was performed three days before the day when culturing supernatant was recovered.

### [4. Measurement of Hyaluronic Acid]

Hyaluronic acid in the culturing supernatant was measured using a hyaluronic acid measurement kit (R&D Co., Cat. No. DY3614) in accordance with a protocol accompanying the kit.

### [5. Statistical Analysis]

A Student's t-test was performed as a significance test, where a result for which P<0.05 (null hypothesis less than 5%) was regarded as having a significant difference.

### [6. Results]

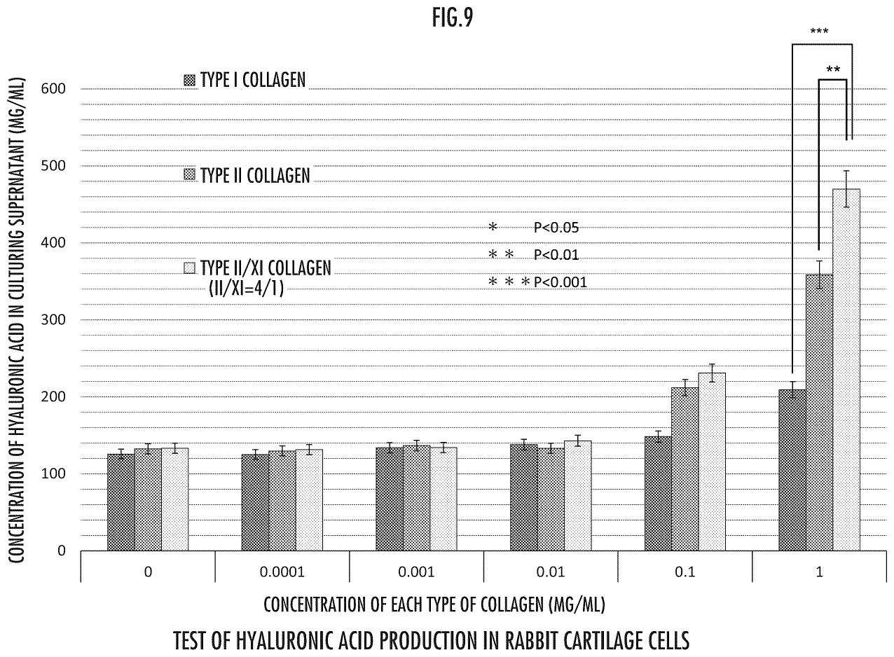

The results are collectively shown in table 1 and FIG. 9.

TABLE 1

| | | Hyaluronic acid concentration in medium (ng/mL) | | | | | | |
|---|---|---|---|---|---|---|---|---|
| | | | | | Type II + | | | Type II + |
| Substances under | | None | Type I | Type II | type XI | Type I | Type II | type XI |
| test and amount | | added | collagen | collagen | collagen | collagen | collagen | collagen |
| thereof added | | 0 | 0.0001 | | | 0.001 | | |
| n1 | | 133 | 119 | 132 | 130 | 139 | 141 | 134 |
| n2 | | 145 | 131 | 128 | 125 | 135 | 135 | 147 |
| n3 | | 126 | 122 | 130 | 141 | 119 | 125 | 129 |
| n4 | | 119 | 141 | 142 | 149 | 144 | 144 | 122 |
| n5 | | 144 | 113 | 117 | 112 | 133 | 139 | 139 |
| AVERAGE | | 133 | 125 | 130 | 131 | 134 | 137 | 134 |
| Standard deviation | | 11 | 11 | 9 | 14 | 9 | 7 | 10 |
| Sample count | | 5 | 5 | 5 | 5 | 5 | 5 | 5 |
| Standard error | | 5.0 | 4.9 | 4.0 | 6.4 | 4.2 | 3.3 | 4.3 |
| Vs. none added | | 100 | 94 | 97 | 99 | 100 | 103 | 101 |
| (% of control) | | | | | | | | |
| Standard error | | 3.8 | 3.7 | 3.0 | 4.8 | 3.1 | 2.5 | 3.2 |
| Level of | Vs. none added | | n.s. | n.s. | n.s. | n.s. | n.s. | n.s. |
| significance | Vs. type I collagen | | | | | | | |
| | Vs. type II collagen | | | | | | | |

| | | Hyaluronic acid concentration in medium (ng/mL) | | | | | | | | |
|---|---|---|---|---|---|---|---|---|---|---|
| | | | | Type II + | | | Type II + | | | Type II + |
| Substances under | | Type I | Type II | type XI | Type I | Type II | type XI | Type I | Type II | type XI |
| test and amount | | collagen | collagen | collagen | collagen | collagen | collagen | collagen | collagen | collagen |
| thereof added | | 0.01 | | | 0.1 | | | 1 | | |
| n1 | | 139 | 143 | 129 | 144 | 181 | 218 | 171 | 350 | 467 |
| n2 | | 129 | 125 | 169 | 126 | 244 | 277 | 197 | 297 | 404 |
| n3 | | 155 | 144 | 142 | 142 | 236 | 201 | 259 | 437 | 482 |
| n4 | | 141 | 133 | 133 | 159 | 223 | 237 | 186 | 361 | 477 |
| n5 | | 127 | 121 | 144 | 171 | 178 | 222 | 234 | 348 | 521 |
| AVERAGE | | 138 | 133 | 143 | 148 | 212 | 231 | 209 | 359 | 470 |
| Standard deviation | | 11 | 10 | 16 | 17 | 31 | 29 | 36 | 50 | 42 |
| Sample count | | 5 | 5 | 5 | 5 | 5 | 5 | 5 | 5 | 5 |
| Standard error | | 5.0 | 4.6 | 7.0 | 7.7 | 13.9 | 12.8 | 16.2 | 22.5 | 18.9 |
| Vs. none added | | 104 | 100 | 107 | 111 | 159 | 173 | 157 | 269 | 352 |
| (% of control) | | | | | | | | | | |
| Standard error | | 3.8 | 3.5 | 5.2 | 5.8 | 10.4 | 9.6 | 12.1 | 16.9 | 14.2 |
| Level of | Vs. none added | n.s. | n.s. | n.s. | n.s. | P < 0.001 | P < 0.001 | P < 0.01 | P < 0.001 | P < 0.001 |
| significance | Vs. type I collagen | | | | | P < 0.01 | P < 0.001 | | P < 0.001 | P < 0.001 |
| | Vs. type II collagen | | | | | | n.s. | | | P < 0.01 |

It was clarified by the results that the collagen-containing composition that contained type XI collagen in addition to type II collagen had an effect for promoting hyaluronic acid production from rabbit cartilage cells. The effect for promoting hyaluronic acid production of the aforementioned composition was more notable than that in cases where type I collagen or type II collagen was used.

### Test Example 6

The utility of the collagen-containing composition that contained the type II collagen and the type XI collagen was evaluated.

Specifically, human testing with able-bodied persons as subjects was implemented as described below.

### [1. Food Products Under Test]

Placebo food product: a food product that is produced using dextrose, particulate silicon dioxide, and calcium stearate as base materials and that is processed into tablets measuring 300 mg each.
Test food product: a food product that is obtained by blending 10 mg of type II/type XI collagen [type II: 80 mass %; type XI: 20 mass %] (derived from salmon nose cartilage) (Linise Co., Inc.) into the tablets produced as placebos.

### [2. Test Subjects]

Volunteers were solicited, and 50 volunteers who satisfied a prescribed standard were randomly assigned to test groups. Table 2 shows the baseline characteristics of the test subjects for each test group.

TABLE 2

| | Placebo group (n = 25) | Test group (n = 25) |
|---|---|---|
| Age | 54.2 ± 1.4 | 54.2 ± 1.2 |
| Male/female | 11/14 | 10/15 |
| BMI (kg/m²) | 23.6 ± 0.5 | 24.6 ± 0.6 |

Average ± SE

### [3. Testing Schedule]

The test subjects were prompted to ingest one tablet per day with cool or lukewarm water without a determined ingestion timing being set. The ingestion period was 16 weeks.

### [4. Evaluation Items]

Sense of discomfort in knee joints was evaluated before and after ingestion of the food products under test. Specifically, five categories of knee joint pain (during daily life, when walking, when ascending or descending stairs, when crouching down and then getting up, and when standing for an extended time) serving as main evaluation items were surveyed by using a visual analogue scale (VAS) method. Additionally, three types of biomarkers (serum CPII, a marker of type II collagen synthesis; urine CTX-II, a marker of type II collagen decomposition; and serum C1, 2C, a marker of type I and type II collagen decomposition) that are known as indices of cartilage turnover in knee joints were measured as auxiliary evaluation items. Analysis of the biomarkers was entrusted to LSI Medience Corp. Additionally, when an action for crouching down and then getting up, an action for squatting, and an action for ascending or descending stairs were requested to be carried out continuously ten times each, the number of times the test subjects were able to carry out the actions were reported.

### [5. Statistical Analysis]

The Wilcoxon signed-rank test was selected for the intra-group comparative analysis. Mann-Whitney U verification was selected for the inter-group comparative analysis. The level of significance for both comparative analyses was 5%.

### [6. Results]

The results are collectively shown in Table 3.

TABLE 3

| | | | | |
|---|---|---|---|---|
| | P-value | | P-value | P-value |
| | before | | before | test |
| Placebo | ingestion | | ingestion | group vs. |
| group | vs. after | Test group | vs. after | placebo |
| (n = 25) | ingestion | (n = 25) | ingestion | group |

Main evaluation items

| | | | | | | | |
|---|---|---|---|---|---|---|---|
| VAS score (mm) | When at rest | Before ingestion | 60.6 ± 1.8 | <0.01 ** | 50.2 ± 4.7 | <0.01 ** | 0.05 |
| | | After ingestion | 35.1 ± 4.9 | | 29.9 ± 5.3 | | 0.48 |
| | | Change amount | −25.5 ± 5.0 | | −20.3 ± 5.8 | | 0.50 |
| | When walking | Before ingestion | 12.0 ± 2.6 | 0.01 ** | 17.0 ± 3.8 | 0.42 | 0.28 |
| | | After ingestion | 18.0 ± 3.4 | | 14.0 ± 3.3 | | 0.40 |
| | | Change amount | 6.0 ± 2.2 | | −3.0 ± 3.6 | | 0.04 * |
| | When ascending | Before ingestion | 30.5 ± 3.1 | 0.75 | 33.3 ± 4.0 | 0.52 | 1.00 |
| | or descending | After ingestion | 28.3 ± 5.3 | | 23.8 ± 2.7 | | 0.68 |
| | stairs | Change amount | −2.3 ± 4.1 | | −9.5 ± 4.7 | | 0.72 |
| | When crouching | Before ingestion | 33.0 ± 4.3 | 1.00 | 25.0 ± 3.2 | 0.82 | 0.85 |
| | down and then | After ingestion | 31.0 ± 4.2 | | 25.0 ± 3.5 | | 1.00 |
| | getting up | Change amount | −2.0 ± 4.1 | | 0.0 ± 4.1 | | 0.86 |
| | When standing | Before ingestion | 21.0 ± 4.3 | 0.63 | 19.0 ± 4.2 | 1.00 | 0.14 |
| | for ten minutes | After ingestion | 15.0 ± 3.8 | | 26.0 ± 3.4 | | 0.28 |
| | | Change amount | −6.0 ± 3.9 | | 7.0 ± 3.4 | | 0.73 |

Auxiliary evaluation items

| | | | | | | | |
|---|---|---|---|---|---|---|---|
| Knee joint | CPII † | Before ingestion | ####±126.6 | 0.28 | ####±121.2 | 0.31 | 0.24 |
| biomarkers | (ng/mL) | After ingestion | ####±121.2 | | ####±108.2 | | 0.25 |
| | | Change amount | −59.4 ± 53.5 | | −91.6 ± 87.5 | | 0.80 |
| | CTX-II | Before ingestion | 268.1 ± 41.9 | 0.23 | 412.3 ± 70.3 | <0.01 ** | 0.09 |
| | (ng/mmol Cr) | After ingestion | 238.8 ± 44.1 | | 316.2 ± 40.9 | | 0.20 |
| | | Change amount | −29.3 ± 52.6 | | −96.1 ± 57.6 | | 0.40 |
| | C1, 2C | Before ingestion | 2.1 ± 0.7 | 0.42 | 2.8 ± 1.0 | 0.02 * | 0.56 |
| | (μg/mL) | After ingestion | 1.5 ± 0.1 | | 2.9 ± 1.0 | | 0.17 |
| | | Change amount | −0.6 ± 0.7 | | 0.1 ± 0.0 | | 0.34 |
| | CTX-II/CPII | Before ingestion | 0.12 ± 0.02 | 0.60 | 0.23 ± 0.09 | 0.04 * | 0.07 |
| | ratio † | After ingestion | 0.10 ± 0.02 | | 0.17 ± 0.03 | | 0.04 * |
| | | Change amount | −0.02 ± 0.02 | | −0.05 ± 0.03 | | 0.34 |
| Number of times | Action for | Before ingestion | 4.4 ± 0.3 | 0.26 | 4.6 ± 0.5 | 0.08 | 0.73 |
| reported that | crouching | After ingestion | 5.0 ± 0.5 | | 5.6 ± 0.4 | | 0.32 |
| action was | down and then | Change amount | 0.6 ± 0.5 | | 1.0 ± 0.4 | | 0.55 |
| able to be | getting up | | | | | | |
| carried out when | Action for | Before ingestion | 6.9 ± 0.7 | 0.42 | 6.8 ± 0.5 | 0.85 | 0.93 |
| action to be | squatting | After ingestion | 6.2 ± 0.7 | | 6.9 ± 0.4 | | 0.48 |
| repeated ten | | Change amount | −0.6 ± 0.8 | | 0.1 ± 0.5 | | 0.45 |
| times was | Action for | Before ingestion | 4.8 ± 0.5 | 0.66 | 4.7 ± 0.6 | 0.21 | 0.96 |
| requested | ascending or | After ingestion | 4.9 ± 0.5 | | 5.6 ± 0.6 | | 0.36 |
| | descending | Change amount | 0.2 ± 0.4 | | 0.9 ± 0.6 | | 0.35 |
| | stairs | | | | | | |

Average ± SE;
† Two test subjects (in test group) who indicated abnormal values were excluded
* P < 0.05;
** P < 0.01

The following was clarified by the results.

### (1)<VAS Score>

In the evaluation of the VAS survey for the main evaluation items, it was confirmed that the scores for a plurality of questions decreased after 16 weeks of ingestion, and a significant difference was indicated in the intra-group comparison. A significant difference (P<0.039) was indicated by the amount of change in the VAS surveys for when walking in the inter-group comparison. For other evaluation items, it was confirmed that the scores in the test group decreased after 16 weeks of ingestion, using the results of the VAS survey for when at rest as a representative example, but no significant difference was indicated in the inter-group comparison because a decrease in scores was indicated for the placebo group as well.

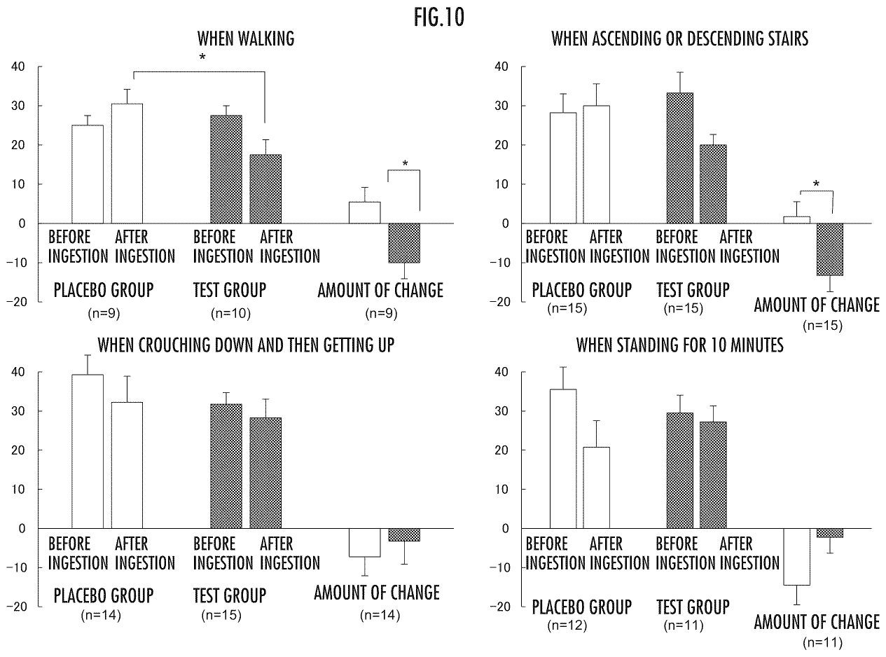

FIG. 10 shows results of hierarchical analysis of the VAS scores for test subjects aged 50 or older, excluding test subjects for whom the score was 10 or lower. There was thereby confirmed to be an inter-group significant difference (P=0.013) in terms of VAS scores for when walking and when ascending or descending stairs after 16 weeks of ingestion. Numerous test subjects for whom the score was 10 or lower were present among test subjects aged under 50, but only a few were present among those aged 50 or older.

### (2)<Joint Cartilage Biomarkers>

In the results of verifying the joint cartilage biomarkers as the auxiliary evaluation items, no large change between before ingestion and after ingestion was confirmed for the serum CPII, which is a biomarker for synthesis of type II collagen. Although the value for the urine CTX-II, which is a biomarker of type II collagen decomposition, was greatly reduced in the test group and a notable intra-group significant difference (P<0.003) was indicated therefor, a downward trend was indicated in the placebo group as well, and no inter-group significant difference for both groups waws confirmed. Additionally, no change between before and after ingestion in the test group was observed for the serum C1, 2C, which is a marker of type I and type II collagen decomposition, and no inter-group significance for both groups was confirmed. The value for the CTX-II/CPII ratio was greatly reduced in the test group, and an intra-group significant difference (P=0.037) was indicated therefor, similarly to the results for the CTX-II. An inter-group significant difference for both groups was also confirmed for the CTX-II/CPII ratio.

### (3)<Survey for Actions Repeated Ten Times>

In the results of surveying actions that were repeated ten times as the auxiliary evaluation items, no large change between groups was confirmed for any of the evaluation items, and no inter-group significant difference for both groups was confirmed.

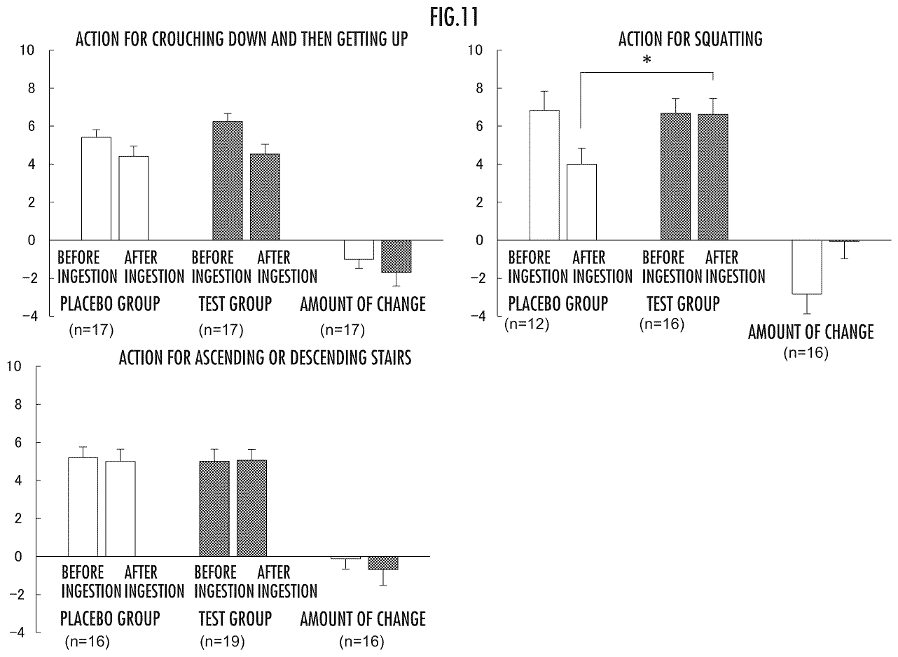

FIG. 11 shows results of hierarchical analysis of test subjects aged 50 or older, excluding test subjects who did not experience any sense of discomfort during the actions. There was thereby indicated to be an inter-group significant difference (P=0.040) after 16 weeks of ingestion in that whereas a decrease was indicated for the number of squats performed in the placebo group, the number of squats performed in the test group slightly increased.

From the results described above, the collagen-containing composition that contained type II collagen and type XI collagen is thought to be effective in protecting knee joints.

1-5. (canceled)

6. A cartilage-protecting composition that contains a collagen-containing composition derived from fish cartilage as an active ingredient, the collagen-containing composition containing type II collagen and type XI collagen, the collagen-containing composition being extracted from the fish cartilage so that the mass ratio of the type II collagen content and the type XI collagen content is 10:1 to 1:10, and that the collagen content per solid fraction is 50 mass % or greater.

7. A knee-joint-protecting composition that contains a collagen-containing composition derived from fish cartilage as an active ingredient, the collagen-containing composition containing type II collagen and type XI collagen, the collagen-containing composition being extracted from the fish cartilage so that the mass ratio of the type II collagen content and the type XI collagen content is 10:1 to 1:10, and that the collagen content per solid fraction is 50 mass % or greater.

8. The composition according to claim 6, wherein the collagen-containing composition is provided with functionality for promoting hyaluronic acid production capabilities in cartilage cells.

9. The composition according to claim 6, wherein the collagen-containing composition is derived from salmon nose cartilage.

10. The composition according to claim 7, wherein the collagen-containing composition is provided with functionality for promoting hyaluronic acid production capabilities in cartilage cells.

11. The composition according to claim 7, wherein the collagen-containing composition is derived from salmon nose cartilage.
